(19) United States
(12) Patent Application Publication
(10) Pub. No.: US 2019/0029644 A1
(43) Pub. Date: Jan. 31, 2019

(54) ULTRASOUND PROBE AND ULTRASOUND DIAGNOSTIC APPARATUS

(71) Applicant: Konica Minolta, Inc., Tokyo (JP)

(72) Inventors: Morio Nishigaki, Fujisawa (JP); Toshiharu Sato, Tokyo (JP); Tatsuya Naito, Tokyo (JP); Takashi Mizuno, Tokyo (JP)

(21) Appl. No.: 16/041,136

(22) Filed: Jul. 20, 2018

(30) Foreign Application Priority Data
Jul. 28, 2017 (JP) 2017-146004

(51) Int. Cl. A61B 8/00; A61B 8/08

(52) U.S. Cl. CPC A61B 8/4494; A61B 8/4466; A61B 8/5207

(57) ABSTRACT

An ultrasound probe includes the following. A plurality of transducers transmit and receive ultrasound arranged in a plurality of rows in a long axis direction, and the rows are aligned in a short axis direction. The ultrasound passes through an acoustic lens. A switching element switches on and off of input of a driving signal to each row of the transducers and output of a receiving signal. The acoustic lens has a shape which guides a plurality of ultrasound beams transmitted and received with each row of transducers to be excluded from one another up to a first predetermined depth.

### BACKGROUND

### 1. Technological Field

The present invention relates to an ultrasound probe and an ultrasound diagnostic apparatus.

### 2. Description of the Related Art

Conventionally, there is an ultrasound diagnostic apparatus in which ultrasound is irradiated on a subject, the reflected ultrasound is received and predetermined signal data processing is performed to generate the ultrasound image of an internal structure of the subject. Such ultrasound diagnostic apparatus is used for a wide variety of purposes such as examination and treatment for medical purposes and examination inside buildings.

The ultrasound diagnostic apparatus not only displays the ultrasound image but is also used when a puncture needle is inserted in a target position while confirming by sight the needle and the position of the target for the following purposes such as collecting a sample of a specific site (target) in the subject, discharging water, inserting and placing medicine or a marker in the specific site. By using the ultrasound image for the above purposes, the treatment on the target in the subject can be performed swiftly, reliably, and easily.

In the ultrasound diagnostic apparatus, transducers which transmit and receive ultrasound are arranged in a one-dimensional or two-dimensional matrix, and the position and direction of sending and receiving ultrasound is scanned (electronic scanning) in a predetermined arranging direction while imaging. The operator such as the physician operates the puncture needle and inserts the puncture needle along the scanning direction (lateral direction). With this, the puncture needle is positioned where continuous imaging is possible from the inserted position of the subject until the puncture needle reaches the target. The puncture needle used to be inserted by being attached to a puncture guide which is an attachment fixed and connected to the ultrasound probe, but lately, the operator usually inserts the puncture needle by freehand.

Therefore, the puncture needle may not always accurately point the first inserting direction or the puncture needle may be bent depending on the internal state or structure of the subject, or the shape of the tip of the puncture needle. As a result, the tip of the puncture needle may be displaced from the region where imaging is possible in the elevation direction orthogonal to the scanning direction, and there were cases where imaging was not possible. Also when the puncture needle is not used and simply the cross-sectional image is obtained, when the operator is not used to using the device, the operator may not be able to perform suitable changes when the position of the ultrasound probe is changed and the imaging range in the elevation direction is finely adjusted. Therefore, much trouble was necessary to obtain the desired image.

There is a known ultrasound diagnostic apparatus with an ultrasound probe in which a plurality of transducers are provided two-dimensionally, there is provided a delay circuit which inputs and outputs transmitting/receiving signals to each of the plurality of transducers in the short axis direction orthogonal to the long axis direction of the array, and a deflection control circuit including a deflection switching switch. The deflection control circuits shift the timing of the transmitting signal and add with a delay to the receiving signal. With this, the ultrasound beam is deflected in the short axis direction, and the puncture needle displaced from the target in the short axis direction is displayed. (Japanese Patent Application Laid-Open Publication No. 2000-139926).

There is also an ultrasound diagnostic apparatus with an ultrasound probe in which a plurality of transducers are arranged two dimensionally and are covered with an acoustic lens with substantially the same curvature entirely. The plurality of transducers are divided in the short axis direction, and some of the divided transducer groups are used in transmitting and receiving so that the ultrasound beam is deflected in the short axis direction and the puncture needle displaced from the target in the short axis direction is displayed. (Japanese Patent Application Laid-Open Publication No. 2016-47191).

However, the ultrasound diagnostic apparatus described in Japanese Patent Application Laid-Open Publication No. 2000-139926 needs a delay circuit for each transducer to deflect the ultrasound beam and the size, cost and heat amount of the apparatus became large.

The ultrasound diagnostic apparatus according to Japanese Patent Application Laid-Open Publication No. 2016-47191 does not need the above-described delay circuit and the size, cost and heat amount of the apparatus does not become large. However, according to the ultrasound diagnostic apparatus according to Japanese Patent Application Laid-Open Publication No. 2016-47191, the acoustic lens with substantially the same curvature is used and the ultrasound beam deflected in the short axis direction and the ultrasound beam not deflected overlap. When the inserted needle tip is included in both ultrasound beams, it may not be possible to determine in which ultrasound beam the position of the puncture needle corresponds to. Therefore, there is a demand to recognize the position of the puncture needle more accurately and easily.

### SUMMARY

The present invention is conceived in view of the above problems, and an object of the present invention is to recognize a position of a recognition target such as a puncture needle in a subject more accurately and easily.

To achieve at least one of the abovementioned objects, according to an aspect of the present invention, an ultrasound probe reflecting one aspect of the present invention is described, the ultrasound probe including, a plurality of transducers which transmit and receive ultrasound arranged in a plurality of rows in a long axis direction, the rows aligned in a short axis direction; an acoustic lens through which the ultrasound passes; and a switching element which switches on and off of input of a driving signal to each row of the transducers and output of a receiving signal, wherein, the acoustic lens has a shape which guides a plurality of ultrasound beams transmitted and received with each row of transducers to be excluded from one another up to a first predetermined depth.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is an entire diagram showing an ultrasound diagnostic apparatus according to an embodiment of the present invention.

FIG. 2 is a block diagram showing an internal configuration of the ultrasound diagnostic apparatus.

FIG. 3 is a diagram showing an example of an arrangement of a transducer in an ultrasound probe.

FIG. 4A is a schematic diagram showing a parallel method of puncture according to an ultrasound guide.

FIG. 4B is a schematic diagram showing an intersecting method of puncture according to an ultrasound guide.

FIG. 5 is a diagram showing a schematic configuration in a short axis direction of the ultrasound probe.

FIG. 6A is a diagram showing directivity with respect to an azimuth angle for first, second, and third transducers.

FIG. 6B is a diagram showing the directivity with respect to the azimuth angle for the first, second, and third transducers with the peak matched.

FIG. 7 is a diagram showing the first, second, and third transducers covered with a normal acoustic lens.

FIG. 8 is a diagram showing a flowchart showing a puncture needle image display process.

FIG. 9A is an upper surface schematic diagram of an ultrasound probe showing a displaced angle when a puncture needle is inserted with a parallel method.

FIG. 9B is a diagram showing a composite image generated using only the second transducer with the parallel method.

FIG. 9C is a diagram showing a composite image generated using the first, second, and third transducers with the parallel method.

FIG. 10A is a diagram showing a composite image generated using only the second transducer with the intersecting method.

FIG. 10B is a diagram showing a composite image generated using the first, second, and third transducer with the intersecting method.

FIG. 11 is a schematic diagram showing an ultrasound beam shape by the first, second, and third transducers in the short axis direction of the ultrasound probe.

### DETAILED DESCRIPTION OF EMBODIMENTS

Hereinafter, one or more embodiments of the present invention will be described with reference to the drawings. However, the scope of the invention is not limited to the disclosed embodiments.

Embodiments of the present invention are described in detail with reference to the drawings.

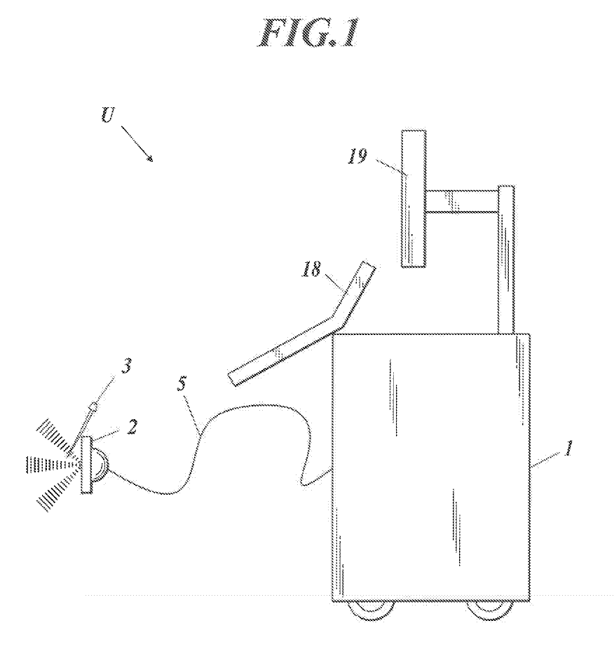

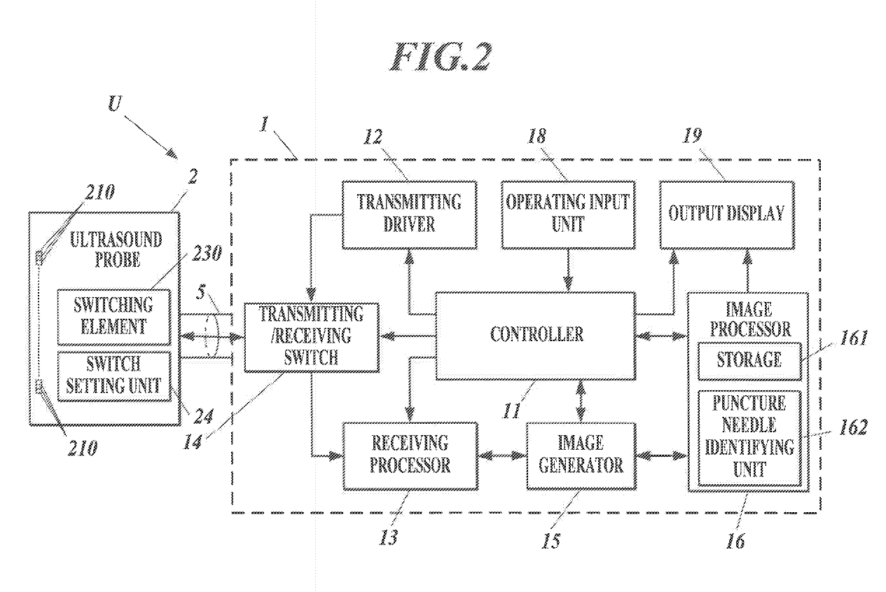

First, the entire configuration of the ultrasound diagnostic apparatus U according to the present embodiment is described with reference to FIG. 1 and FIG. 2. FIG. 1 shows an entire diagram of the ultrasound diagnostic apparatus U according to the present embodiment. FIG. 2 is a block diagram showing an internal configuration of the ultrasound diagnostic apparatus U.

As shown in FIG. 1, the ultrasound diagnostic apparatus U includes an ultrasound diagnostic apparatus main body 1, an ultrasound probe 2 connected to the ultrasound diagnostic apparatus main body 1 through a cable 5, and a puncture needle 3 which is a treatment tool as a recognition target.

Here, the puncture needle 3 has a hollow long needle shape, and is inserted in the subject in an angle determined by freehand operated by an operator such as a physician. The puncture needle 3 can be changed with a needle which has the suitable width, length, or tip shape according to the collected site (target) of the subject such as the patient or the type or amount of the inserted medicine. According to the ultrasound diagnostic apparatus U, an attaching portion as an attachment which guides the puncture needle 3 in the puncture direction or a guiding portion which is provided fixed to the ultrasound probe 2 and which guides the puncture needle 3 in the puncture direction can be provided.

The ultrasound diagnostic apparatus main body 1 is provided with an operating input unit 18 and an output display 19. As shown in FIG. 2, the ultrasound diagnostic apparatus main body 1 includes, in addition to the above, a controller 11, a transmitting driver 12, a receiving processor 13, a transmitting/receiving switch 14, an image generator 15, and an image processor 16. Based on the input operation from outside on the input device such as the keyboard or the mouse of the operating input unit 18, the controller 11 outputs a driving signal to the ultrasound probe 2 to output ultrasound or obtains the receiving signal regarding the ultrasound reception from the ultrasound probe 2, performs various processes, and displays the result such as the display screen of the output display 19 as necessary.

The controller 11 includes a CPU (Central Processing Unit), an HDD (Hard Disk Drive), and RAM (Random Access Memory). The CPU reads various programs stored in the HDD, loads the programs in the RAM, and centrally controls the operation of each unit of the ultrasound diagnostic apparatus U according to the program. The HDD stores the control programs and various processing programs to operate the ultrasound diagnostic apparatus U and various setting data. Specifically, the HDD stores a puncture needle image display program to perform a later-described puncture needle image display process. The programs and setting data described above can be stored in a rewritable state in an auxiliary storage using a nonvolatile memory such as a flash memory including a SSD (Solid State Drive) other than the HDD. The RAM is a volatile memory such as a SRAM or DRAM and provides a work memory space to the CPU to store temporary data.

The transmitting driver 12 outputs the driving signal supplied to the ultrasound probe 2 according to the control signal input from the controller 11 and the ultrasound probe 2 emits the ultrasound. For example, the transmitting driver 12 includes a clock generating circuit, a pulse width setting unit, a pulse generating circuit, and a delay circuit. The clock generating circuit is a circuit which generates the clock signal to determine the transmitting timing and the transmitting frequency of the pulse signal. The pulse width setting unit sets the waveform (shape), voltage amplitude and pulse width of the transmitting pulse output from the pulse generating circuit. The pulse generating circuit generates the transmitting pulse as the driving signal based on the setting of the pulse width setting unit and outputs the pulse to a different line path for each transducer 210 of the ultrasound probe 2. The delay circuit counts the clock signal output from the clock generating circuit and when the set delay time passes, the pulse width generating circuit generates the transmitting pulse and outputs the pulse to each wire path.

The receiving processor 13 is a circuit which obtains the receiving signal input from the ultrasound probe 2 according to the control of the controller 11. For example, the receiving processor 13 includes an amplifier, an A/D (Analog to Digital) conversion circuit, and a phase adding circuit. The amplifier is a circuit which amplifies the receiving signal in response to the ultrasound received by each transducer 210 of the ultrasound probe 2 at a predetermined amplifying rate. The A/D conversion circuit is a circuit which converts the amplified receiving signal to digital data at a predetermined sampling frequency. The phase adding circuit is a circuit which provides a delay time to the A/D converted receiving signal for each line path corresponding to each transducer 210 to adjust the time phase and adds the above (phase adding) to generate sound ray data.

Based on the control by the controller 11, the transmitting/receiving switch 14 switches between transmitting the driving signal from the transmitting driver 12 to the transducer 210 when the transducer 210 emits (transmits) the ultrasound and outputting the receiving signal to the receiving processor 13 when the signal receiving the ultrasound emitted from the transducer 210 is obtained.

The image generator 15 generates the diagnostic image based on the receiving data of the ultrasound. The image generator 15 detects (envelope detections) sound ray data input from the receiving processor 13 to obtain the signal and performs logarithm amplifying, filtering (for example, low pass, smoothing, etc.), and emphasizing process as necessary. The image generator 15 generates frame image data regarding B (Brightness) mode display for the tomographic image showing a two-dimensional configuration in the cross-section including the transmitting direction (depth direction of subject) of the brightness signal according to the signal strength and the scanning direction (lateral direction, long axis direction of the two-dimensional array of the transducers 210) of the ultrasound transmitted by the ultrasound probe 2 as one of the diagnostic images. Here, the image generator 15 is able to adjust the dynamic range and perform the gamma correction of the display. The image generator 15 is able to include the dedicated CPU and RAM used in the image generating. Alternatively, in the image generator 15, the dedicated hardware configuration regarding image generating may be formed on the substrate (ASIC (Application-Specific Integrated Circuit)) or may be formed with a FPGA (Field Programmable Gate Array). Alternatively, the image generator 15 can be a configuration in which the process regarding the image generating is performed in the CPU and RAM of the controller 11.

The image processor 16 includes a storage 161 and a puncture needle identifying unit 162. The storage 161 stores a predetermined number of frames of recent diagnostic image data (frame image data) in the unit of frames to be processed in the image generator 15 and used in the real time display or the like. For example, the storage 161 is a volatile memory such as a DRAM (Dynamic Random Access Memory). Alternatively, the storage 161 is a non-volatile memory which is rewritable at a high speed. The diagnostic image data stored in the storage 161 is read according to control of the controller 11, transmitted to the output display 19, and output outside the ultrasound diagnostic apparatus U through the communicating unit (not shown). Here, when the display of the output display 19 is a television, a DSC (Digital Scan Converter) is provided between the storage 161 and the output display 19, and the data is output after the scanning format is converted.

The puncture needle identifying unit 162 generates image data to identify the position of the puncture needle 3, performs suitable processes on the image data to extract and identify the needle portion image of the position including the tip of the puncture needle 3, and colors the needle portion image of the extracted puncture needle 3. The puncture needle identifying unit 162 can be used together with the CPU and the RAM of the image processor 16 or can include a dedicated CPU and RAM. Alternatively, the puncture needle identifying unit 162 performs various processes with the CPU and the RAM of the controller 11. The puncture needle identifying unit 162 stores the tip position information of the identified puncture needle 3 as history.

As the method to identify the position of the puncture needle 3, for example, Japanese Patent No. 6123458 describes obtaining the difference or correlation of frames from the plurality of frames of ultrasound image data to generate motion evaluation information showing the evaluation of motion, calculating the movement velocity of the tip of the puncture needle, detecting the position of the tip of the puncture needle from the movement velocity of the tip of the puncture needle and the movement evaluation information, and identifying the position of the puncture needle including the tip. The position of the puncture needle 3 including the tip can be identified by estimating the position of the tip in the future based on the movement history of the tip of the puncture needle 3, and detecting the tip using the estimated position as reference. Alternatively, the outline may be detected by input on the operating input unit 18 so that the operator selects the outline from among the candidates obtained first and the outline similar to the selected outline may be used to detect the position of the puncture needle with the estimated position as reference.

The operation input unit 18 includes a press button switch, a keyboard, a mouse or trackball or a combination of the above. The operating input unit 18 converts the input operation by the user to the operating signal and inputs the signal to the ultrasound diagnostic apparatus main body 1.

The output display 19 includes a display screen using any of the following display methods such as an LCD (Liquid Crystal Display), an organic EL (Electro-Luminescent) display, an inorganic EL display, a plasma display, a CRT (Cathode Ray Tube) display, and a driver to drive the above. The output display 19 generates a control signal output from the CPU 15, generates the driving signal of the display screen (display pixels) according to the image data generated in the image processor 16, and displays the menu and status of the ultrasound diagnosis or the measurement data based on the received ultrasound on the display screen. The output display unit 19 includes a separate LED (Light Emitting Diode) lamp and displays whether the power is turned on or off.

The operating input unit 18 and the output display unit 19 can be formed as one with the casing of the ultrasound diagnostic apparatus main body 1 or can be externally attached through a RGB cable, a USB (Universal Serial Bus) cable, or an HDMI (High-Definition Multimedia Interface). When the ultrasound diagnostic apparatus main body 1 is provided with an operation input terminal and a display output terminal, conventional peripheral devices for operation and display can be connected to these terminals.

The ultrasound probe 2 functions as an acoustic sensor which emits ultrasound (here, about 1 to 30 MHz) to a subject such as a live body, receives an echo of the emitted ultrasound reflected on the subject, and converts the reflected wave (echo) to electric signals.

The ultrasound probe 2 includes a plurality of transducers 210 which transmits and receives ultrasound, switching elements 230 corresponded to each transducer 210, and a switch setting unit 24. Here, the ultrasound probe 2 emits ultrasound from outside (body surface) to inside the subject and receives the reflected wave. The ultrasound probe 2 further includes those with shapes and sizes to be used inserted inside a digestive track, a blood vessel, or inside the body cavity. The operator presses the surface of the ultrasound probe 2 which transmits and receives ultrasound, that is, the surface in the emitting direction of the ultrasound from the transducer 210 to the subject with predetermined pressure to operate the ultrasound diagnostic apparatus U and performs ultrasound diagnosis.

The number of transducers composing the transducers 210 can be set freely. According to the present embodiment, a linear scanning type electronic scanning probe is employed as the ultrasound probe 2. Alternatively, an electronic scanning type or mechanical scanning type can be employed. A linear scanning type, a sector scanning type or a convex scanning type may also be employed.

The transducers 210 are a plurality of transducers provided with a piezoelectric element including a piezoelectric body and an electrode provided on both edges in which charge appears due to deforming (extending and contracting) of the piezoelectric body.

When the voltage pulse as the driving signal is supplied to each of the plurality of transducers 210, the piezoelectric body of the transducer to which the voltage pulse is supplied is deformed (extends/contracts) according to the electric field generated in the piezoelectric body and the ultrasound is emitted. The emitted ultrasound is emitted to the position and direction according to the position, direction of the transducers 210 included in the predetermined number of transducer rows to which the voltage pulse is supplied, focusing direction of the emitted ultrasound and the magnitude of the shift (delay) in the timing. When the ultrasound (reflected wave in the subject) of the predetermined frequency band enters the transducer 210, the thickness of the piezoelectric body changes (oscillates) by the sound pressure and the charge is generated according to the change amount. The conversion to the electric signal is performed according to the charge amount and the result is output as the receiving signal.

The switch setting unit 24 stores the setting of the transmitting and the receiving sequence of the transducer 210 to perform the transmitting and receiving of the ultrasound in the short axis direction of the two-dimensional array of the transducers 210 (elevation direction) and switches the on and off of the switching element 230 corresponding to the transducers 210 according to the setting. The transmitting and receiving sequence of the transducer 210 is described later.

The cable 5 includes a connector (not shown) in each edge to connect to the ultrasound diagnostic apparatus main body 1 and the ultrasound probe 2. The ultrasound probe 2 is detachable from the ultrasound diagnostic apparatus 1 because of this cable 5. The cable 5 can be formed as one with the ultrasound probe 2.

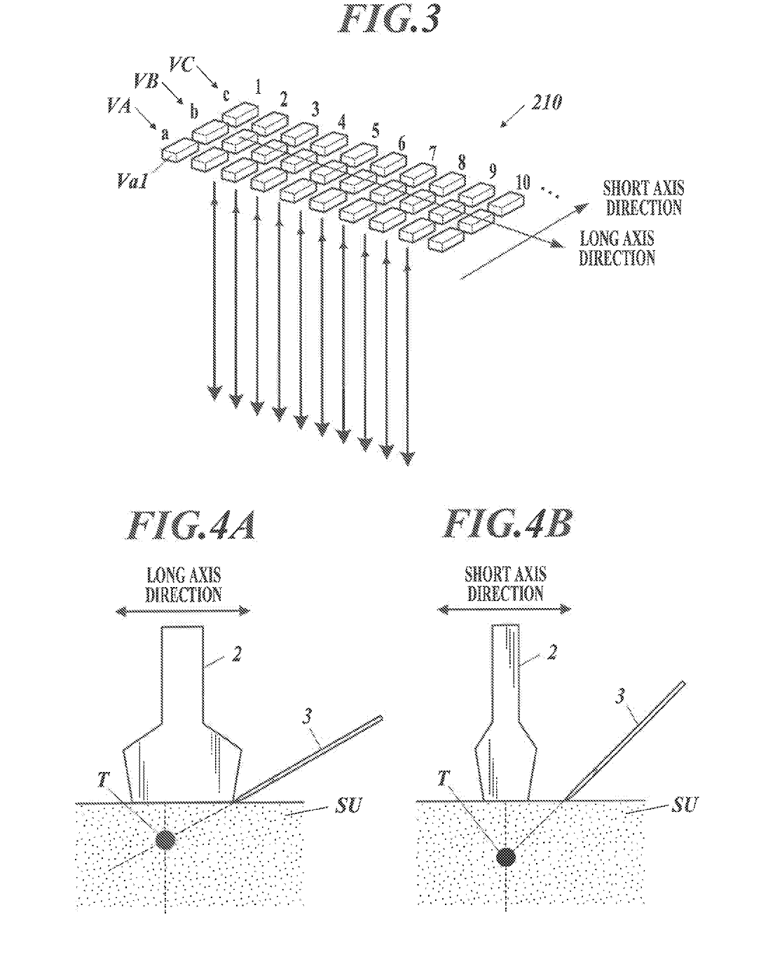

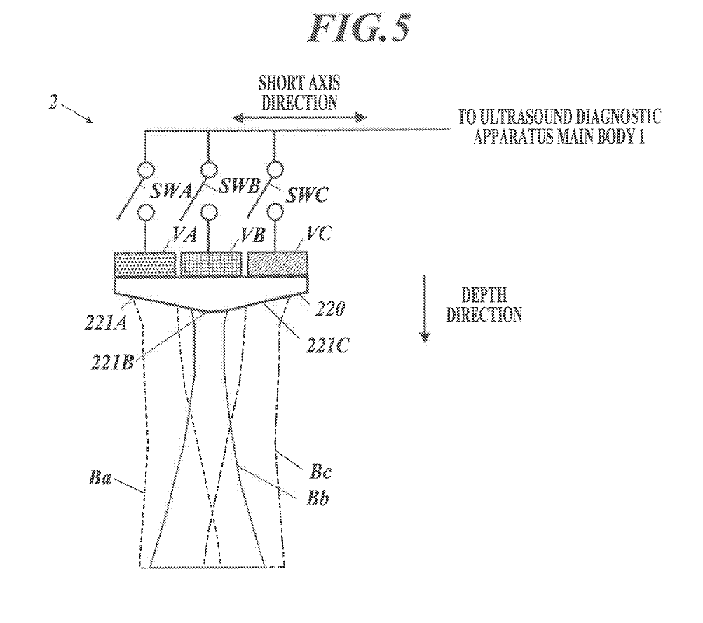

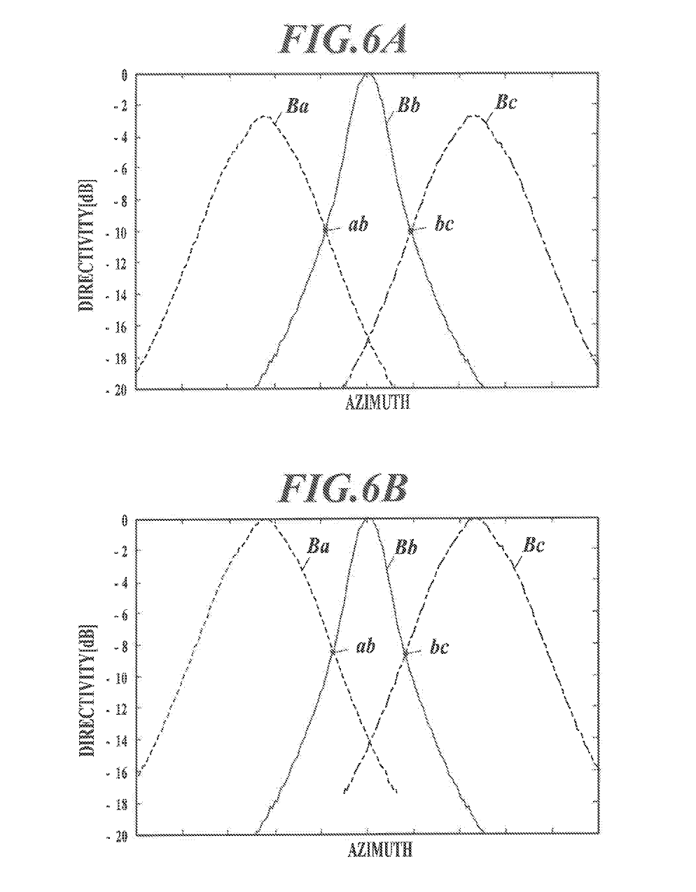

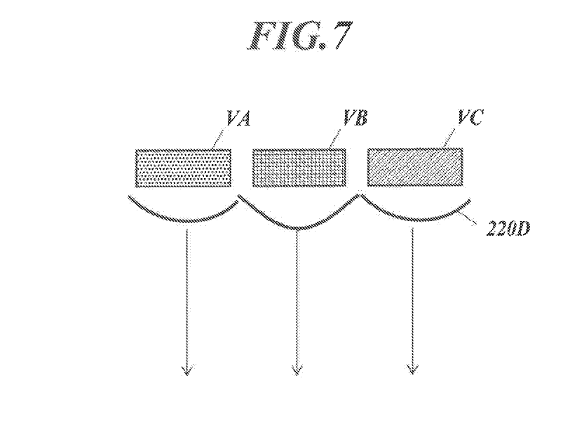

Here, the detailed configuration and operation of the ultrasound probe 2 is described with reference to FIG. 3 to FIG. 7. FIG. 3 is a diagram showing an example of an array of the transducers 210 in the ultrasound probe 2. FIG. 4A is a schematic diagram showing a parallel method for the puncture under the ultrasound guide. FIG. 4B is a schematic diagram showing an intersecting method for the puncture under the ultrasound guide. FIG. 5 is a diagram showing a schematic configuration in the short axis direction of the ultrasound probe 2. FIG. 6A is a diagram showing directivity with respect to the azimuth of the transducers VA, VB, and VC. FIG. 6B is a diagram showing the directivity with respect to the azimuth of the transducers VA, VB, and VC matching the peak. FIG. 7 is a diagram showing the transducers VA, VB, and VC covered by a normal acoustic lens 220D.

As shown in FIG. 3, in the ultrasound diagnostic apparatus U, the transducers 210 are a plurality of transducers arranged in a matrix in a two-dimensional surface (does not have to be a plane) defined by a predetermined lateral direction (scanning direction) and an elevation direction orthogonal to the lateral direction. Normally, the array of the transducers 210 in the lateral direction is larger than the array of the transducers 210 in the elevation direction, and the lateral direction is the long axis direction and the elevation direction is the short axis direction. The transducer 210 includes transducer groups in three rows (row a, b, c) in the short axis direction and a plurality of columns of transducers (column 1, 2, etc.) in the long axis direction. To simplify explanation, the transducer group of row a is referred to as transducer VA, and similarly the transducer groups of the rows b, c, are referred to as transducers VB, VC. One transducer in column x and row y is referred to as transducer Vxy.

When the normal B mode (tomographic) image is generated, the ultrasound is transmitted and received sequentially shifting the transducer driven in the long axis direction using the transducer VB of the row b.

Here, the parallel method and the intersecting method are described as the method for puncture of the puncture needle 3 under the ultrasound guide is described with reference to FIG. 4A and FIG. 4B.

As shown in FIG. 4A, the parallel method is a method in which the puncture needle 3 is inserted parallel to the long axis direction towards the target T such as obtaining tissue by puncture of the subject SU. As shown in FIG. 4B, the intersecting method is a method in which the puncture needle 3 is inserted in the direction orthogonal to the long axis direction toward the target T of the subject SU. The parallel method and the intersecting method are used according to the purpose of use. The method is determined by the site where the subject is punctured or the purpose of puncture, but the method may be selected according to how experienced the operator is.

According to the parallel method, when the puncture needle is inserted, the puncture needle punctures the subject SU from the long axis edge of the ultrasound probe, and the puncture needle is inserted towards the deep position in the tomographic face formed by the puncture needle of the long axis in one row corresponding to the transducer VB. When the puncture needle 3 is displaced from the tomographic face in the short axis direction, the puncture needle 3 is not illustrated in conventional ultrasound diagnostic apparatuses.

According to the intersecting method, the puncture needle 3 is inserted diagonally in the subject SU from the short axis side of the ultrasound probe and the puncture needle is inserted in the target T right under the ultrasound probe. In the conventional intersecting method, when a normal ultrasound probe is used, the puncture needle 3 puncturing the body surface is not displayed in the ultrasound image even when the puncture needle 3 is inserted quite deep. The puncture needle first appears in the ultrasound image near the target T. Therefore, it is difficult to know whether the inserted puncture needle 3 is headed in the correct direction.

According to the present embodiment, in both the parallel method and the intersecting method, the puncture needle 3 is caught in a wide region. Therefore, in addition to the frame of the ultrasound image by the transducer VB, the frame of the ultrasound image by the transducer VA and the frame of the ultrasound image by the transducer VC can be obtained at the same time of day.

As shown in FIG. 5, the ultrasound probe 2 includes, in the short axis direction viewed from the long axis edge, an acoustic lens 220, transducers VA, VB, VC, switches SWA, SWB, SWC of the switching element 230 corresponding to the above. An acoustic matching layer positioned between the acoustic lens 220 and the transducers VA, VB, VC, and a backing material positioned opposite to the ultrasound emitting direction of the transducers VA, VB, VC are not illustrated.

The acoustic lens 220 is an aspherical lens focusing the ultrasound beam (transmitting ultrasound) which is output from the transducers VA, VB, VC. The acoustic lens 220 includes a lens 221A through which the ultrasound beam Ba emitted from the transducer VA passes, a lens 221B through which the ultrasound beam Bb emitted from the transducer VB passes, and a lens 221C through which the ultrasound beam Bc emitted from the transducer VC passes.

The switch SWA is a switch which can separately turn on and off the input of the driving signal to the transducers of the transducer VA and the output of the receiving signal from the transmitting/receiving switch 14 through the switch setting unit 24 and cable 5. The switch SWB is a switch which can separately turn on and off the input of the driving signal to the transducers of the transducer VB and the output of the receiving signal from the transmitting/receiving switch 14 through the switch setting unit 24 and cable 5. The switch SWC is a switch which can separately turn on and off the input of the driving signal to the transducers of the transducer VC and the output of the receiving signal from the transmitting/receiving switch 14 through the switch setting unit 24 and cable 5.

According to the above-described embodiment, the transducers VA, VB, and VC are positioned so that there is no overlapping portion or space until a certain depth between the ultrasound beams Ba, Bb, and Bc.

The short axis width of the transducer VB has a width which can stand normal ultrasound scanning. The lens 221B of the acoustic lens 220 which covers the transducer VB includes the ability to form the beam which can be used in normal ultrasound scanning. The short axis width of the transducers VA and VC can be smaller than the transducer VB, but has a width to sufficiently obtain the reflected wave (echo) of the inserted puncture needle 3. Preferably, the lenses 221A and 221C of the acoustic lens 220 which cover the transducers VA and VC have an aspherical shape to have a larger curvature radius than the lens 221B, but it is possible to use a diagonal flat shape. A flat shape which is not diagonal is not impossible, but a diagonal shape is preferable considering fusion with the ultrasound beam Bb of the transducer VB. The lens shape of the lens 221B may be an aspherical shape so that connection with the lenses 221A and 221C is smooth as long as problems do not occur in normal ultrasound scanning (for example, side lobe becoming large).

In order to accurately catch the position of the puncture needle 3, preferably, the positions covered by the ultrasound beams Ba, Bb, Bc transmitted and received by the transducers VA, VB, VC are exclusive among each other. Being exclusive, the reflecting wave (echo) from the puncture needle 3 is only included in the reflecting wave from one among the transducers VA, VB, VC. Therefore, the discrimination becomes easy.

However, the directivity of the ultrasound beam has a shape with a gentle sloping base, and the base of the ultrasound beams Ba, Bb, Bc overlap. Therefore, as shown in FIG. 5, the above are not completely exclusive. FIG. 6A shows overlap of the ultrasound beams Ba, Bb, Bc in a certain depth. In FIG. 6A, the horizontal axis shows the azimuth of the short axis direction, the vertical axis shows the directivity, and shows the beam shape of the ultrasound beams Ba, Bb, Bc by the transducers VA, VB, VC. In order to easily discriminate in which ultrasound beam the reflecting wave of the puncture needle 3 is included, preferably, it is known from experience that the point where the adjacent ultrasound beam becomes the same level (points ab, be in FIG. 6A), is −6 [dB] to −12 [dB] from the peak of the transmitting or receiving ultrasound beam.

Preferably, regarding the peak of the ultrasound beam, the peak of the ultrasound beam Bb by the transducer VB is used as the reference. For example, compared to the short axis width of the transducer VB, when the short axis width of the transducers VA, VC is smaller, it is assumed that the height of the ultrasound beams Ba, Bc become lower compared to the height of the peak of the ultrasound beam Bb. The difference of the height of the ultrasound beam can be obtained in advance by calculation, and the difference can be corrected using the calculated value. FIG. 6B shows the height of the peaks of the directivity of the transducers VA, VC shown in FIG. 6A matched to the height of the directivity of the transducer VB. When the difference of the sensitivity among the transducers is large, the needle position can be accurately caught by the above. The difference in the sensitivity among the transducers occur depending on the depth, and the correction can be performed when the difference is large.

Here, the suitable shape of the acoustic lens 220 according to the present embodiment is described. First, as shown in FIG. 7, a configuration in which the transducers VA, VB, VC are covered with an acoustic lens 220D with a shape in which normal short axis lenses are connected is considered. In this case, the ultrasound beams from each of the transducers VA, VB, VC have a parallel directivity as illustrated with an arrow. However, the ultrasound beam of the transducer VB becomes thinner from the transducer VB towards the focal point. That is, the ultrasound beams by the transducer VA and VC need to cover (have directivity) the left and the right of the thinning ultrasound beam of the transducer VB. If there is a space (to be accurate a zone in which sensitivity of both ultrasound beams is low) in the directivity of the ultrasound beam of the transducer VA and the ultrasound beam of the transducer VB, or the directivity of the ultrasound beam of the transducer VC and the ultrasound beam of the transducer VB, it is difficult to catch the puncture needle 3 when the puncture needle 3 is positioned in the portion. Therefore, preferably, the shape of the acoustic lens is shaped so that the ultrasound beam of the transducer VA and the ultrasound beam of the transducer VC are deflected toward the inside.

When the ultrasound beam of the transducers VA, VC is focused in a shallow position near the transducers VA, VC, the puncture needle 3 inserted by the intersecting method does not come into the ultrasound beam, that is, the puncture needle 3 cannot be caught. Considering the above, preferably, the ultrasound beam of the transducers VA, VC is deflected but the focal position is in a deep position or not focused.

When the acoustic lens with a lens shape so that the ultrasound beam of the transducer VA and the ultrasound beam of the transducer VC are deflected toward the inside is used, as the depth becomes deeper, the ultrasound beams of the transducers VA, VC overlap with the center ultrasound beam of the transducer VB, and it is not possible to discriminate the position of the puncture needle 3. Therefore, the deflection angle should not be too large, and preferably, the ultrasound beams of the transducers VA, VB, VC are separated so that the needle position can be identified to a depth where there is no problem in examination.

The depth to which the ultrasound beams are separated depends on the diagnosis site. For example, when the high frequency linear probe is used as the ultrasound probe 2, preferably, the separating is to be possible up to 25 to 30 [mm]. The shape of the acoustic lens 220 which matches the above condition is the aspherical lens shape of the acoustic lens 220 as shown in FIG. 5. In the acoustic lens 220, the curvature is sharp in the lens 221B corresponding to the transducer VB (curvature radius is small) and the curvature is gentle in the lenses 221A, 221C corresponding to the transducers VA and VC (curvature radius is large).

As shown in FIG. 5, in the parallel method, when the acoustic lens 220 is used, the switch SWB is turned on and the switches SWA, SWC are turned off, and the transmitting and receiving of the ultrasound is performed using the transducer VB. The operation in such case is no different from the transmitting and receiving of ultrasound in a conventional ultrasound diagnostic apparatus as described above.

If the switch SWA is turned on and the switches SWB, SWC are turned off, the ultrasound is transmitted and received by the transducer VA. Since the lens 221A corresponding to the transducer VA is a substantial diagonal aspherical shape, the transmitting and receiving beam of the ultrasound is deflected to the transducer center side and the intersecting point of the transmitting/receiving beam and the center line becomes a far position compared to the focal point of the lens 221B corresponding to the transducer VB. When the curvature is set in the lenses 221A and 221C, preferably, the curvature is set so that the focus is near the intersecting point. The transmitting/receiving beam of the ultrasound formed by the acoustic lens 220, the transducers VA, VB, VC do not overlap to a predetermined depth, and there is no space (blind spot of sensing). According to the present embodiment, with the parallel method, since the tomographic image using the transducers VA, VC is formed in addition to the tomographic image using the transducer VB, the puncture needle displaced from the tomographic image face of the transducer VB can be caught.

As shown in FIG. 4B, when the ultrasound probe 2 according to the present embodiment is used in the intersecting method, the target T in the subject SU is in the tomographic image of the transducer VB. In the tomographic image of the transducer VA (or the transducer VC) the inserted puncture needle 3 can be captured faster compared to when the normal ultrasound probe is used. As an example, when inserted toward a target at a depth of 1 cm with an inserting angle of 45 degrees, if the ultrasound beam width of the transducer VB is 1.8 mm, the puncture needle can be first seen about 1 mm before the target with the normal ultrasound probe whereas the puncture needle can be confirmed from about 4 mm before with the ultrasound probe 2 according to the present embodiment. As described above, according to the intersecting method of the present embodiment, the position of the puncture needle 3 can be confirmed much before the position of the target T, and the puncture operation becomes easier.

Here, with reference to FIG. 3, the transmitting and receiving sequence of the transducer 210 according to the present embodiment is described. As described above, according to the configuration using the acoustic lens 220, the transducers VA, VB, VC, and switches SWA, SWB, SWC, in order to obtain the reflecting wave (echo) of the puncture needle 3 outside the ultrasound beam Bb formed by the transducer VB, the ultrasound is transmitted and received using the transducers VA, VB. In this case, scanning (ultrasound transmitting and receiving) can be performed in the following order, for example, transducers V1a, V1b, V1c, V2a, V2b, V2c, V3a, V3b, V3c, . . . . Such scanning sequence is stored in the switch setting unit 24.

However, in this case, since the number of transmitting and receiving increases multiplied by three times, the frame rate of the B mode tomographic image display reduces to three thirds. The scanning of the transducers VA, VC to capture the puncture needle 3 is thinned, and for example, the scanning can be performed in the following order to prevent reduction of the frame rate, transducers V1a, V1b, V1c, V2b, V3a, V3b, V3c, V4b, V5a, V5b, V5c, . . . . According to the above description, one transducer is used for scanning in the long axis direction to simplify description, but actually, a plurality of transducers are used to form the transmitting/receiving beam in the long axis direction. Other than the above, it is possible to use well-known parallel reception in the long axis direction as the method to increase the frame rate.

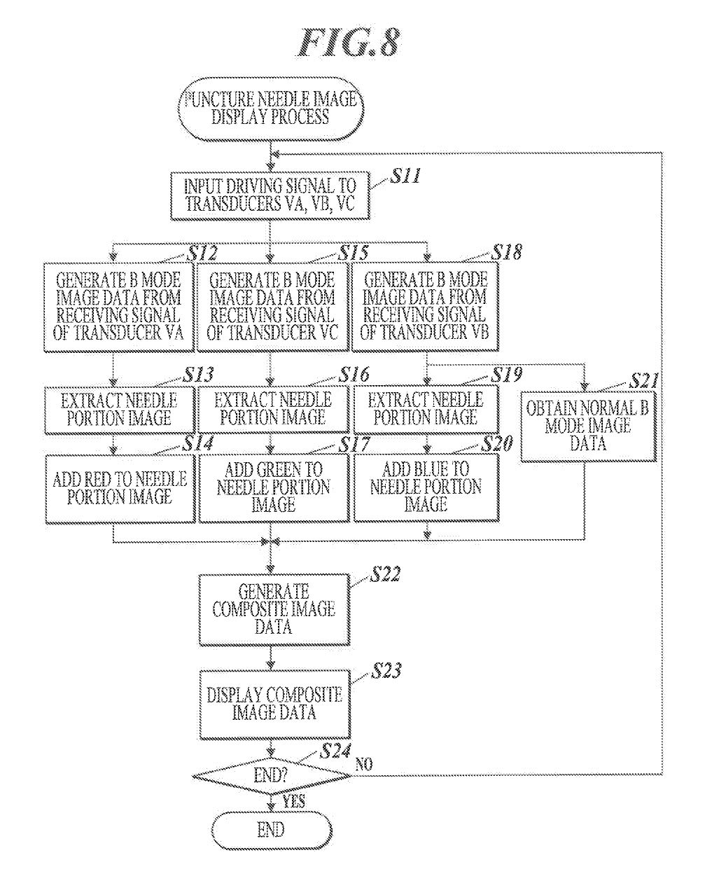

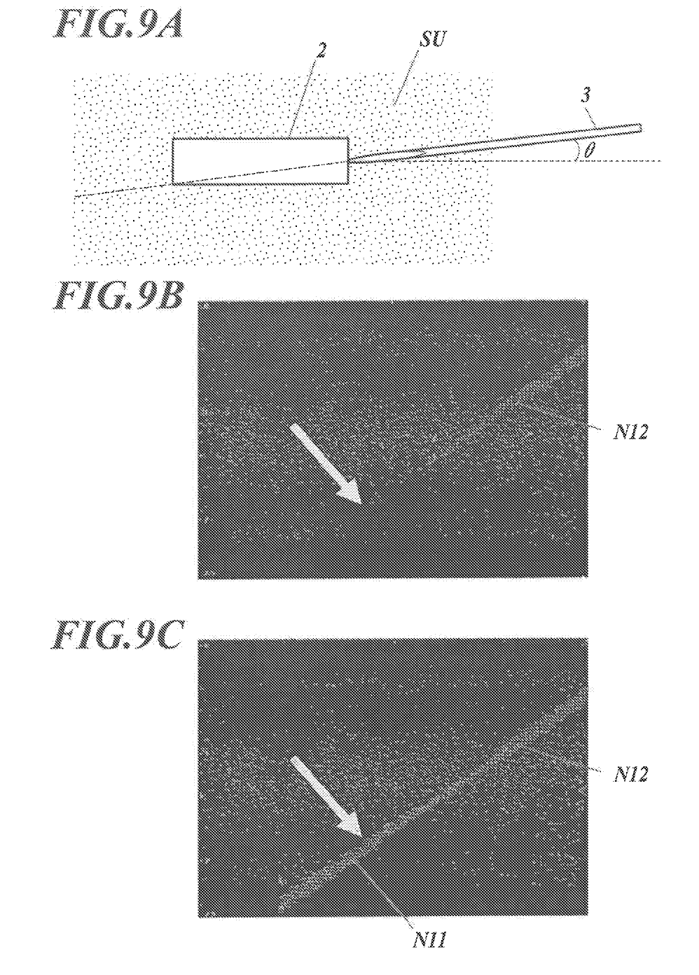

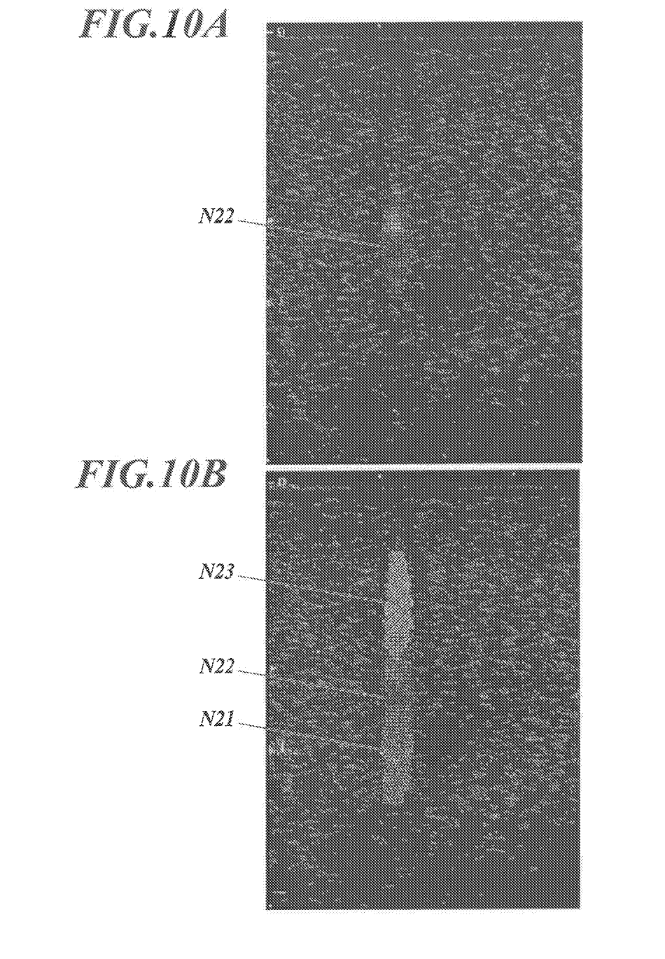

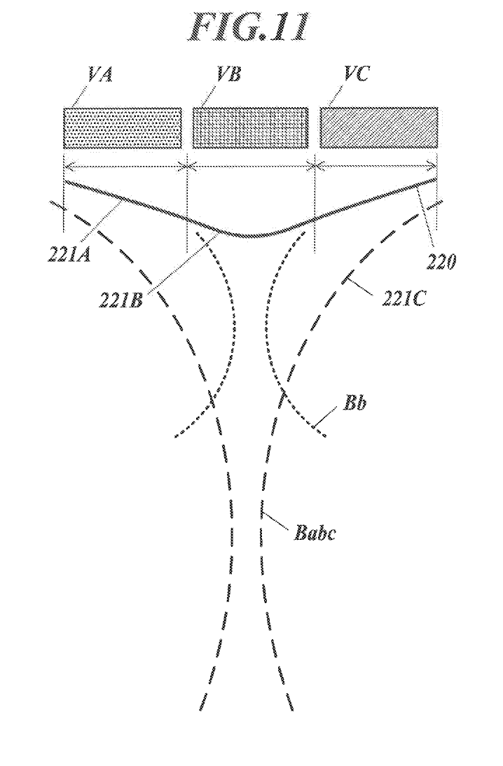

Next, with reference to FIG. 8 to FIG. 11, the operation of the ultrasound diagnostic apparatus U is described. FIG. 8 is a flowchart showing a puncture needle image display process. FIG. 9A is a schematic diagram of an upper surface of the ultrasound probe 2 showing a displaced angle θ of the puncture needle 3 in the parallel method. FIG. 9B is a diagram showing an ultrasound image generated using only the transducer VB in the parallel method. FIG. 9C is a diagram showing a composite image generated using the transducers VA, VB, VC in the parallel method. FIG. 10A is a diagram showing an ultrasound image generated using only the transducer VB in the intersecting method. FIG. 10B is a diagram showing a composite image generated using the transducers VA, VB, VC in the intersecting method. FIG. 11 is a schematic diagram showing an ultrasound beam Babc of the transducers VA, VB, VC in the short axis direction of the ultrasound probe 2.

The puncture needle image display process performed in the ultrasound diagnostic apparatus U is described with reference to FIG. 8. The puncture needle image display process is a process to assist the puncture process by displaying the B mode tomographic image of the puncture needle 3 in the target live when the operator such as the physician inserts the puncture needle 3 in the target T as the target to obtain tissue of the subject SU.

For example, as preparation, the operator such as the physician waits in an examination room where the ultrasound diagnostic apparatus U is provided. The patient as the subject SU enters the examination room and lies on the bed. Preparations to perform the puncture process using the puncture needle 3 is finished. Then, the ultrasound diagnostic apparatus U receives from the operator through the operating input unit 18 input of various setting information such as the frame rate in the puncture needle image display process and the instruction to perform the puncture needle image display process. Such operation acts as a trigger and the controller 11 performs the puncture needle image display process according to the puncture needle image display program stored in the ROM.

First, the controller 11 controls the transmitting driver 12 to start generating the driving signal. The controller 11 controls the transmitting/receiving switch 14 to switch the switching element 230 according to the transmitting/receiving sequence stored in the switch setting unit 24. With this, the driving signal is input to the transducers of the transducers VA, VB, VC so that the transmitting ultrasound is emitted. The reflecting ultrasound (echo) is received. The controller 11 controls the transmitting/receiving switch 14 so that the receiving processor 13 obtains the receiving signal (step S11). The receiving signal obtained in step S11 is obtained in the order that the receiving signal for each frame at the same time corresponding to the transducers VA, VB, VC responds to the transmitting/receiving sequence.

Then, the controller 11 controls the image generator 15 to generate the B mode image data of 1 frame from the receiving signal corresponding to the transducer VA input from the receiving processor 13 in step S11 (step S12). Then, the controller 11 controls the puncture needle identifying unit 162 to extract the needle portion image of the puncture needle 3 from the B mode image data corresponding to the transducer VA generated in step S12 (discard portion other than needle portion image) (step S13). Then, the controller 11 controls the puncture needle identifying unit 162 and colors the needle portion image of the image data generated in step S13 with red showing the transducer VA (step S14).

The controller 11 controls the image generator 15 to generate the B mode image data of one frame from the receiving signal corresponding to the transducer VC input from the receiving processor 13 in step S11 (step S15). The B mode image data generated in step S15 is a frame with the same time of day as the B mode image data generated in step S12. Then, the controller 11 controls the puncture needle identifying unit 162 to extract the needle portion image of the puncture needle 3 from the B mode image data corresponding to the transducer VC generated in step S15 (step S16). Then, the controller 11 controls the puncture needle identifying unit 162 to color the needle portion image of the image data generated in step S16 with green showing the transducer VC (step S17).

The controller 11 controls the image generator 15 to generate the B mode image data of one frame from the receiving signal corresponding to the transducer VB input from the receiving processor 13 in step S11 (step S18). The B mode image data generated in step S18 is a frame with the same time of day as the B mode image data generated in steps S12, S15. Then, the controller 11 controls the puncture needle identifying unit 162 to extract the needle portion image of the puncture needle 3 from the B mode image data corresponding to the transducer VB generated in step S18 (step S19). Then, the controller 11 controls the puncture needle identifying unit 162 and colors the needle portion image of the image data generated in step S19 with blue showing the transducer VB (step S20).

In steps S14, S17, and S20, a different color is used to show from which transducer VA, VB, VC the needle portion image is obtained. The combination of the colors in steps S14, S17, and S20 is not limited to the above, and for example, a gradation such as green-blue-purple can be used. Further, the expression to be able to discriminate each needle portion image can be changed to a different expression. For example, the expression to be able to discriminate the different needle portion image can be a different saturation and brightness, or a different flashing and interval. Various types of expressions may be combined.

In steps S11, S12, S15, S18, processes corresponding to the first input various setting information is performed. During the puncture needle image display process, input can be made by the operator through the operating input unit 18 to suitably change the various setting information. The operator can input on the operating input unit 18 the expression (color) of the needle portion image in the steps S14, S17, S20 as the various setting information.

After step S18 is executed, the controller 11 controls the image generator 15 to obtain the normal B mode image data of one frame generated in step S11 (step S21). Then, the controller 11 controls the image processor 16 to combine the red needle portion image generated in step S14, the green needle portion image generated in step S17, the blue needle portion image generated in step S20, and the B mode image data of one frame obtained in step S21, and generates the combined image data of one frame (step S22). Then, the controller 11 controls the image processor 16 to display the combined image data of one frame generated in step S22 on the output display 19 (step S24).

Then, the controller 11 determines whether the end instruction of the puncture needle image display process is input from the operator through the operating input unit 18 (step S24). When the process does not end (step S24; NO), the process advances to step S11. When the process ends (step S24; YES), the puncture needle image display process ends.

Next, with reference to FIG. 9A to FIG. 10B, an image example of the puncture needle image display process in the parallel method and the intersecting method is described.

With reference to FIG. 9A to FIG. 9C, the image generated and displayed in the puncture needle image display process when the puncture process is performed in the parallel method is described. First, as shown in FIG. 9A, as the parallel method, an example of inserting the puncture needle 3 in the target below the ultrasound probe 2 when the ultrasound probe 2 held against the surface of the subject SU is viewed from above is described. The displacement of the puncture needle 3 in the axis direction from the central line C in the long axis direction of the ultrasound probe 2 is shown with the displacement angle θ of the puncture needle 3. In FIG. 9A, the operator starts inserting the puncture needle 3 from the long axis edge of the ultrasound probe 2. The position of the puncture needle 3 is matched at this point, and as the inserting progresses, the position of the puncture needle 3 is displaced from the target due to the displacement angle θ.

The puncture needle 3 is inserted from the state shown in FIG. 9A and in the puncture needle image display process, only the transducer VB is used. The ultrasound image including the needle portion image N12 of the puncture needle 3 as shown in FIG. 9B is displayed. That is, the composite image in the step S11 (transmitting and receiving only with the transducer VB) and steps S18 to S23 is displayed. In the ultrasound image shown in FIG. 9B, blue is expressed with a “cross hatching” and the same can be said for FIG. 9C, FIG. 10A, and FIG. 10B. The ultrasound image shown in FIG. 9B shows the puncture needle 3 including the region of the ultrasound beam of the transducer VB with blue.

With the same state and the same time as the transmitting and receiving of the ultrasound image shown in FIG. 9B, the composite image shown in FIG. 9C is obtained by the puncture image display process using the transducers VA, VB, VC. In FIG. 9C, red is expressed with “dots”, and the same can be said for FIG. 10B. In the composite image shown in FIG. 9B, the needle portion image N12 of the puncture needle 3 including the region of the ultrasound beam of the transducer VB is shown with blue. As shown with the arrow, the needle portion image N11 of the puncture needle 3 included in the region of the ultrasound beam of the transducer VA is shown with red.

In FIG. 9B, the arrow is shown in the same position as the arrow of FIG. 9C, and the puncture needle 3 cannot be confirmed by sight in the position of the arrow. As described above, in the parallel method, only by adding the needle portion image of the puncture needle 3 captured with the transducer VA (or VC) to the B mode image used in the transducer VB, it is possible to clearly show whether the puncture needle 3 is displaced from the center line C and in which direction the puncture needle 3 is displaced.

Next, as shown in FIG. 4B, in the intersecting method, the example of inserting the puncture needle 3 towards the target below the ultrasound probe 2 from the direction in an angle 45 degrees from the surface of the subject SU is described. From this state, the puncture needle 3 is inserted, and similar to the conventional method, with the puncture needle image display process, using only the transducer VB, the ultrasound image using the needle portion image N22 of the puncture needle 3 as shown in FIG. 10A is displayed. That is, the ultrasound image displayed by performing the process of step S11 (transmitting and receiving only with the transducer VB), and steps S18 to S23.

Then, in the same state and at the same time as the transmitting and receiving of the ultrasound image shown in FIG. 10A, the composite image shown in FIG. 10B is obtained by the puncture needle image display process using the transducers VA, VB, VC. In FIG. 10B, green is expressed with “hatching”. In the composite image shown in FIG. 10B, the needle portion image N22 of the puncture needle 3 included in the region of the ultrasound beam of the transducer VB is shown with blue, and on both sides of the ultrasound tomographic image of the transducer VB in the short axis direction, the needle portion image N21 of the puncture needle 3 included in the region of the ultrasound beam of the transducer VA is shown with red, and the needle portion image N23 of the puncture needle 3 included in the region of the ultrasound beam of the transducer VC is shown with green. Therefore, a portion of the shallow puncture needle 3 shown with green on the transducer VC side, a portion of the puncture needle 3 shown with blue near the depth of the target corresponding to the transducer VB, and a portion of the deep puncture needle 3 shown with red on the transducer VA side is continuously confirmed visibly as one object.

As described above, with the intersecting method, only by adding the needle portion image of the puncture needle 3 captured with the transducers VA, VC to the ultrasound image using the transducer VB, the range that the puncture needle 3 can be seen is enlarged. Therefore, the depth of the puncture needle 3 can be shown clearly with color and can be easily understood. With this, safe puncture operation can be performed.

Preferably, the puncture needle image display process uses all of the transducers VA, VB, VC in the ultrasound image generating, and the deep illustration abilities are enhanced.

A method is well-known to illustrate a comparatively shallow portion using the transducer in the center in the short axis direction among a plurality of transducers arranged two-dimensionally and to illustrate a portion relatively deep using all of the transducers in the short axis direction. As shown in FIG. 11, in the ultrasound probe 2, the focal distance of the ultrasound beam Babc generated using all of the transducers VA, VB, VC in the short axis direction and the acoustic lens 220 is longer than the focal distance of the ultrasound beam Bb generated using the transducer VB in the center in the short axis direction and the lens 221B of the acoustic lens 220.

When a relatively deep portion is illustrated with the ultrasound probe 2, preferably, the lenses 221A and 221C corresponding to the transducers VA, VC are set so that the ultrasound beam Bac passes the center of the entire short axis direction at the depth desired to be illustrated (position of the ultrasound beam Bb of the transducer VB) and have the curvature so that the ultrasound beam Babc is focused at the depth desired to be illustrated. With this, the deep portion illustrating ability is enhanced. The depth desired to be illustrated is deeper than the predetermined depth where the ultrasound beams Ba, Bb, Bc corresponding to the transducers VA, VB, BC are exclusive from each other.

According to the present embodiment, the ultrasound probe 2 includes a plurality of transducers VA, VB, VC which are aligned in the short axis direction arranged in three rows a, b, c in the long axis direction and which transmit and receive ultrasound, an acoustic lens 220 which passes ultrasound, and a switching element 230 (switches SWA, SWB, SWC) which switches the input of the driving signal to the transducers VA, VB, VC of each row a, b, c and the output of the receiving signal. The acoustic lens 220 includes a shape to form the plurality of ultrasound beams Ba, Bb, Bc transmitted and received with the transducers VA, VB, VC in each row exclusive from each other to a first predetermined depth. The first predetermined depth is a depth depending on the diagnosed site, and may be 25 to 30 [mm], for example.

Therefore, by combining the B mode image data of each row a, b, c, generated by using the transducers VA, VB, VC of the rows a, b, c switched accordingly, the position of the puncture needle 3 inserted in the subject as the treatment tool can be accurately and easily acknowledged in the composite image.

The acoustic lens 220 includes a shape so that the plurality of ultrasound beams Ba, Bb, Bc transmitted and received with the transducers VA, VB, VC in each row are formed exclusive from each other and without spaces up to the first predetermined depth. Therefore, the region where the puncture needle 3 cannot be seen in the composite image combining the B mode image data of the rows a, b, c can be reduced.

The acoustic lens 220 includes lenses 221A, 221B, 221C corresponding to the transducers VA, VB, VC of the rows a, b, c. The lenses 221A, 221C corresponding to the transducers VA, VC of the rows a, c other than the center in the short axis direction have an aspherical shape to form the ultrasound beams Ba, Bc deflected to the ultrasound beam Bb side formed by the lens 221B corresponding to the transducer VB of the row b at the center in the short axis direction. Therefore, the plurality of ultrasound beams Ba, Bb, Bc transmitted and received with the transducers VA, VB, VC of the rows a, b, c up to a first predetermined depth can be formed easily exclusive from each other and without space.

The lens 221A, 221C corresponding to the transducers VA, VC of the rows a, c other than the center in the short axis direction have a curvature focusing to the center in the short axis direction at the second predetermined depth. The second predetermined depth is a depth deeper than the first predetermined depth and is the depth where high quality illustration is desired. Therefore, the illustrating ability is enhanced in the composite image data using all of the transducers VA, VB, VC at the desired second predetermined depth.

The ultrasound diagnostic apparatus U includes an ultrasound probe 2, a transmitting driver 12 which outputs the driving signal to the transducers VA, VB, VC of the rows a, b, c, of the ultrasound probe 2 through switching of the switching element 230, the receiving processor 13 which obtains the receiving signal corresponding to the transducers VA, VB, VC of the rows a, b, c from the ultrasound probe 2 through the switching of the switching element 230, an image generator 15 which generates the B mode image data corresponding to each row a, b, c, from the receiving signal corresponding to each row a, b, c, and an image processor 16 which generates the composite image data combining the plurality of generated B mode image data. Therefore, the position of the puncture needle 3 in the subject can be acknowledged accurately and easily in the composite image of the B mode image data using the transducers VA, VB, VC in each row a, b, c.

The controller 11 displays the composite image data on the output display 19. Therefore, the position of the puncture needle 3 in the subject can be acknowledged accurately and easily in the displayed composite image.

The image processor 16 extracts the needle portion image of the puncture needle 3 from the three sets of B mode image data corresponding to the transducers VA, VB, VC of each row a, b, c and displays each extracted needle portion image with different colors so as to be able to discriminate the rows a, b, c. The image processor combines the B mode image data corresponding to the transducer VB among the three sets of B mode image data and the needle portion image displaying each row a, b, c with different colors so as to be able to discriminate the rows a, b, c to generate the composite image data. Therefore, since the colors displaying the needle portion images are different, the position of the puncture needle 3 in the subject can be acknowledged accurately and easily. Specifically, in the parallel method, according to the difference in the colors displaying the needle portion image, whether the puncture needle 3 is displaced and the direction that the puncture needle 3 is displaced (direction to the transducer VA side or the transducer VC side) can be acknowledged by sight accurately and easily. In the intersecting method, according to the needle portion image with different colors, the puncture needle 3 can be largely confirmed by sight, and according to the difference in the colors displaying the needle portion image, the depth of the puncture needle 3 can be acknowledged accurately and easily.

The description of the above-described embodiments show one example of a suitable ultrasound diagnostic apparatus according to the present invention, and the present invention is not limited to the above.

For example, according to the present embodiment, the ultrasound diagnostic apparatus U generates and displays the B mode image data as the ultrasound image data, but the configuration is not limited to the above. The ultrasound diagnostic apparatus U can generate and display the tomographic image data in another mode as the ultrasound image data.

According to the present embodiment, the ultrasound probe 2 in which three rows including a plurality of transducers arranged in the long axis direction are aligned in the short axis direction is shown, but the present invention is not limited to the above. The divided number (transducer number) in the short axis direction can be increased such as five rows, seven rows, etc. in the short axis direction or a plurality of transducers can be used at once to increase the number of regions exclusive from each other.

According to the present embodiment, the portion image of the puncture needle 3 as the treatment tool is extracted from the ultrasound image data and colored as the target to be acknowledged, but the present invention is not limited to the above. For example, a site such as a blood vessel in the subject can be the target to be acknowledged, and the portion image of the site can be extracted from the ultrasound image data and colored (with an expression so as to be able to discriminate the rows) or a pseudo (simple) three-dimensional display is possible.

The detailed configuration and the detailed operation of the units composing the ultrasound diagnostic apparatus U according to the present embodiment can be suitably modified without leaving the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2017-146004 filed on Jul. 28, 2017 is incorporated herein by reference in its entirety.

What is claimed is:

1. An ultrasound probe comprising:
a plurality of transducers which transmit and receive ultrasound arranged in a plurality of rows in a long axis direction, the rows aligned in a short axis direction;
an acoustic lens through which the ultrasound passes; and
a switching element which switches on and off of input of a driving signal to each row of the transducers and output of a receiving signal,
wherein, the acoustic lens has a shape which guides a plurality of ultrasound beams transmitted and received with each row of transducers to be excluded from one another up to a first predetermined depth.

2. The ultrasound probe according to claim 1, wherein the acoustic lens has a shape which guides a plurality of ultrasound beams transmitted and received with each row of transducers to be excluded from one another and to substantially not have space between each other up to the first predetermined depth.

3. The ultrasound probe according to claim 1, wherein,
the acoustic lens includes a lens corresponding to each row of transducers, and
the lens corresponding to a row of transducers other than a center in the short axis direction has an aspherical shape which guides an ultrasound beam to be deflected toward an ultrasound beam passing a lens corresponding to the row of transducers at a center in the short axis direction.

4. The ultrasound probe according to claim 3, wherein the lens corresponding to the row of transducers other than the center in the short axis direction has a curvature to focus to the center in the short axis direction at a second predetermined depth which is deeper than the first predetermined depth.

5. An ultrasound diagnostic apparatus comprising:
the ultrasound probe according to claim 1;
a transmitter which outputs a driving signal to each row of transducers in the ultrasound probe through switching of the switching element;
a receiver which obtains a receiving signal corresponding to each row of transducers from the ultrasound probe through the switching of the switching element;
a first hardware processor which generates ultrasound image data corresponding to each row from the receiving signal corresponding to each row, and generates composite image data combining the plurality of generated ultrasound image data.

6. The ultrasound diagnostic apparatus according to claim 5, further comprising a second hardware processor which displays the composite image data on the display.

7. The ultrasound diagnostic apparatus according to claim 5, wherein, the first hardware processor extracts a partial image of a target from the plurality of ultrasound image data, uses a separate distinguishable expression for each row in the extracted partial image, and combines one of the plurality of ultrasound image data with the partial image including the separate distinguishable expression for each row to generate composite image data.

8. The ultrasound diagnostic apparatus according to claim 7, wherein, the expression is at least any one among displayed color, saturation, brightness, and flashing.
